(19) United States
(12) Patent Application Publication
(10) Pub. No.: US 2008/0286341 A1
(43) Pub. Date: Nov. 20, 2008

(54) Buffered coated nicotine containing products

(76) Inventors: Sven-Borje Andersson, Odakra (SE); Gunnar Bergengren, Bjuv (SE); Bengt Bosson, Helsingborg (SE); Andreas Hugerth, Bjarreo (SE); Fredrik Nicklasson, Bjarreo (SE); Roland Olsson, Helsingborg (SE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/803,865

(22) Filed: May 16, 2007

(30) Foreign Application Priority Data
May 16, 2007 (SE) SE 0701177-8

(51) Int. Cl. A61K 9/28; A61K 31/44; A61K 9/68; A61P 43/00

(52) U.S. Cl. 424/440; 424/441; 514/343

(57) ABSTRACT

Coated oral dosage forms for the delivery of nicotine in any form to a subject by rapid intraoral delivery of nicotine comprising at least one core, nicotine in any form and/or a nicotine mimicking agent, at least one coating layer and optionally at least one or more other additives, wherein said at last one coating layer is buffered, whereby is used at least one amino acid as buffering agent. Also contemplated are a method for the delivery of nicotine in any form, a method for the reduction of the urge to smoke or use tobacco as well as a method for producing said coated product and use of the same for obtaining a rapid intraoral uptake of nicotine.

### TECHNICAL FIELD

This invention relates to coated oral dosage forms for intraoral delivery of nicotine to a subject. The coated oral dosage forms comprise one or more amino acids as buffer. Also contemplated are a method and a system for delivering nicotine as well as use and production of said coated oral dosage forms.

### BACKGROUND OF THE INVENTION

Tobacco Dependence and Reduction Thereof

In recent years, with the recognition of the harmful effects of tobacco smoking, there have been numerous campaigns and programs by governmental agencies and various health groups and other interested organisations to disseminate information about the adverse health effects resulting from tobacco smoking. Moreover, and as a result of this recognition of the harmful effects, there have been many programs directed to attempts in reducing smoking incidence.

Nicotine is an organic compound and is the principal alkaloid of tobacco. Nicotine is the chief addictive ingredient in the tobacco used in cigarettes, cigars, snuff and the like. Nicotine is also an addictive drug, though, and smokers characteristically display a strong tendency to relapse after having successfully stopped smoking for a time. Nicotine is the worlds second most used drug, after caffeine from coffee and tea.

The main problem with tobacco smoking is its enormous implications on health. It is estimated that smoking related diseases cause some 3-4 million deaths per year. According to Centers for Disease Control and Prevention. Around 500,000 persons in USA die each year as a result of tobacco use, see United States, 1995 MMWR 1997; 46:1217-1220. In fact, excessive smoking is now recognised as one of the major health problems throughout the world. This grim consequence of tobacco smoking has urged many medical associations and health authorities to take very strong actions against the use of tobacco.

Even though tobacco smoking is decreasing in many developed countries today it is hard to see how the societies could get rid of the world's second most used drug. The incidence of smoking is still rising in many countries, especially in less developed countries.

The most advantageous thing a heavy smoker can do is to stop smoking completely or at least reduce his smoking. Experience shows, however, that most smokers find this extremely difficult since, mostly, tobacco smoking results in a dependence disorder or craving. The WHO has in its International Classification of Disorders a diagnosis called Tobacco Dependence. Others like the American Psychiatric Association call the addiction Nicotine Dependence. It is generally accepted that these difficulties to stop smoking result from the fact that those heavy smokers are dependent on nicotine. The most important risk factors are, however, substances that are formed during the combustion of tobacco, such as carbon monoxide, carcinogenic tar products, N-nitrosamines, aldehydes, and hydrocyanic acid.

Effects of Nicotine

The administration of nicotine can give satisfaction and the usual method is by smoking, either by smoking eg a cigarette, a cigar or a pipe. However, smoking has health hazards and it is therefore desirable to formulate an alternative way of administering nicotine in a pleasurable manner that can be used to facilitate withdrawal from smoking and/or used as a replacement for smoking.

When smoking a cigarette, nicotine is quickly absorbed into the smoker's blood and reaches the brain within around ten seconds after inhalation. The quick uptake of nicotine gives the consumer a rapid satisfaction, or kick. The satisfaction, then, lasts during the smoking time of the cigarette and for a period of time thereafter. The poisonous, toxic, carcinogenic, and addictive nature of smoking has provided efforts for methods, compositions and devices, which help in breaking the habit of smoking cigarettes.

Nicotine is an addictive poisonous alkaloid C₅H₄NC₄H₇NCH₃, derived from the tobacco plant. Nicotine is also used as an insecticide.

Nicotine Replacement Products

One way to reduce smoking is to provide nicotine in a form or manner other than by smoking and some products have been developed to fulfil this need. Nicotine containing formulations are currently the dominating treatments for tobacco dependence.

The successes in achieving reduction in the incidence of smoking have been relatively poor using presently known products. The present state of the art involves both behavioural approaches and pharmacological approaches. More than 80% of the tobacco smokers who initially quit smoking after using some behavioural or pharmacological approach to singly reduce smoking incidence generally relapse and return to the habit of smoking at their former rate of smoking within about a one year's period of time.

As an aid for those who are willing to stop smoking there are several ways and forms of nicotine replacement products available on the market. Several methods and means have been described for diminishing the desire of a subject to use tobacco, which comprises the step of administering to the subject nicotine or a derivative thereof as described in eg U.S. Pat. No. 5,810,018 (oral nicotine-containing spray), U.S. Pat. No. 5,939,100 (nicotine-containing micro-spheres) and U.S. Pat. No. 4,967,773 (nicotine-containing lozenge).

Nicotine-containing nose drops have been reported (Russell et al., British Medical Journal, Vol. 286, p. 683 (1983); Jarvis et al., Brit. J. of Addiction, Vol. 82, p. 983 (1987)). Nose drops, however, are difficult to administer and are not convenient for use at work or in other public situations. Ways of administrating nicotine by way of delivering directly into the nasal cavity by spraying is known from U.S. Pat. No. 4,579,858, DE 32 41 437 and WO/93 127 64. There may, though, be local nasal irritation with use of nasal nicotine formulations. The difficulty in administration also results in unpredictability of the dose of nicotine administered.

The use of skin patches for transdermal administration of nicotine has been reported (Rose, in Pharmacologic Treatment of Tobacco Dependence, (1986) pp. 158-166, Harvard Univ. Press). Nicotine-containing skin patches that are in wide use today can cause local irritation and the absorption of nicotine is slow and affected by cutaneous blood flow.

Also, inhaling devices resembling a cigarette are known for uptake of nicotine vapours as suggested in U.S. Pat. No. 5,167,242.

One of the most successful approaches to date in reducing the incidence of smoking relies upon nicotine containing chewing gum that is designed to reduce smoking withdrawal symptoms. The reported success rate is approximately twice that of placebo. The use of the nicotine gum suffers from several problems eg that it has been found that the nicotine containing gum does not sufficiently rapidly satisfy the craving that most smokers experience. One successful product that is used as a smoking substitute and/or as a smoking cessation aid and which is based on nicotine is the chewing gum Nicorette®. This product was one of the first nicotine replacement forms that was approved by the Food and Drug Administration (FDA) and is still one of the most used nicotine replacement products. Nicorette® chewing gum has been on the market in about 80 countries for several years. In this chewing gum the nicotine is present in the form of a complex with an insoluble cation-exchanger (polacrilex) that is dispersed in a gum base. The nicotine is slowly released from the gum due to chewing and will reach similar plasma levels as when smoking a cigarette after about 30 minutes depending on the chewing technique, ie slow or active. Patents related to this product are, eg U.S. Pat. Nos. 3,877,468, 3,901,248 and 3,845,217.

WO 98/23165 discloses a chewing gum wherein nicotine may be in a non-buffered coating. This concept may provide rapid release of the nicotine from the coated chewing gum, but not a sufficiently rapid buccal uptake of the nicotine. The fraction of the released nicotine that is not immediately absorbed will be flushed down in the gastrointestinal (G.I.) tracts by the saliva, thereby possibly causing hiccups and other G.I. side effects. Once absorbed by the G.I. route this swallowed nicotine will be subjected to first pass metabolism.

WO 00/13662 discloses a chewing gum for systemic, oral administration of an active whereby said active is administered by the chewing gum composition in a bi-phasic manner. The bi-phasic delivery is obtained by the gum matrix as such, not from a coating.

WO 00/19977 discloses a substantially moisture free and possibly coated chewing gum for delivery of an active. The nicotine is preferably encapsulated. The possible coating is not buffered.

WO 00/35296 discloses a coated nicotine-containing chewing gum with a non-buffered coating.

WO 02/102357 discloses a coated nicotine-containing chewing gum, where at least one coating layer is buffered. This gum provides improved transmucousal absorption of nicotine in the oral cavity. Thereby is achieved more of a cigarette-like sense of satisfaction and a more rapid reduction of the urge to smoke. The buffers proposed in WO 02/102357 though possess off-notes. Therefore one or more flavouring agents need be added to the gum in order to cover the off-note taste. Further, the drying time for the layers of the coated gum of WO 02/102357 is unacceptably long.

The present invention presents a solution to inter alia the above problems.

U.S. Pat. No. 5,733,572 discloses gas filled micro-spheres, which, as stated in a long and non-substantiated laundry list on actives and excipients, may further comprise nicotine and certain amino acids, the latter though not for buffering purposes, but for achieving a depot action effect. To date nothing has been disclosed on the utility of amino acids as buffers in coated nicotine-containing pharmaceutical formulations.

### SUMMARY OF THE INVENTION

When formulating a medical product intended to dissolve in the oral cavity the organoleptic characteristics are essential. Beside, in many cases there is a need to obtain optimal pH in the oral cavity in order to achieve a sufficiently rapid sufficient uptake of the active ingredient. By using a buffering agent in the product said pH can be adjusted. However, the number of pharmaceutically appropriate buffering agents is limited and some of the most commonly used buffering agents possess distinct off-notes. Therefore, one or more flavoring and/or taste-masking agents are usually added to the formulation to cover the off-notes. Moreover, flavoring agents are also used in the formulation to accomplish a product with pleasant taste. The possibility of using a buffering agent with no or comparably mild off-taste, facilitates the formulation work and reduces the complexity of the flavoring and/or taste-masking process.

It has surprisingly been found that many of the amino acids as buffering agents possess no intrinsic taste and consequently, the use of these excipients in products for oral uptake has been found to be beneficial by the present inventors. More particularly, there is provided a coated pharmaceutical product for intraoral delivery of nicotine comprising at least one buffered or non-buffered core, nicotine in any form and optionally a nicotine mimicking agent, at least one coating layer and optionally one or more other additive(s), wherein said at least one coating layer is buffered, whereby is used at least one amino acid as buffering agent.

Another important criterion for choosing a suitable buffering compound is its toxicity. Many of the common amino acids can be classified as harmless since they occur in large amounts, several grams daily, in common nutrition.

Other advantages in using amino acids as buffers in nicotine-containing formulations encompass lack of unpleasant smell and that many of the amino acids of interest have monographs in both USP/NF and Ph.Eur and that many of them are found in the FDA-list of inactive ingredients.

When using both an active agent and a buffering agent in a product there may emerge a need for keeping these two ingredients apart to avoid any unwanted chemical reaction. These ingredients may hence eg be placed in separate layers. The drying time of such different layers may be extremely lengthy and not within a reasonable process timeframe. Numerous different buffering agents were evaluated to find a buffering agent providing for an acceptable drying time, but none gave an acceptable outcome until, surprisingly, the introduction of amino acids to the manufacturing process for the present formulations resulted in an acceptable drying time. As stated above amino acids have outstanding characteristics for buffering purposes, whereby problems with both off-notes and long drying times are avoided.

In view of the foregoing disadvantages known in the art when trying to deliver nicotine to a subject so as to obtain a rapid transmucosal uptake of nicotine in the oral cavity of the subject the present invention provides a new and improved product, systems and methods for obtaining a rapid transmucosal uptake of nicotine in the oral cavity of the subject, while avoiding off-notes from the buffer used and while obtaining acceptable drying times for coating layers of the product.

An object of the present invention is to provide an efficient and effective product, as well as methods and systems for a rapid uptake of nicotine in a subject and to avoid the disadvantages of such previously known products and methods.

Thus, the present invention provides a method for delivering nicotine in any form to a subject comprising administering to a subject said coated oral dosage form containing nicotine in any form into the oral cavity of the subject and allowing the nicotine in any form in the coated oral dosage form product to be released in the saliva in the oral cavity and absorbed into the systemic circulation of the subject as well as a method for producing said coated oral dosage form.

The coated oral dosage form is intended for release of nicotine primarily in the oral cavity. The coated oral dosage form is preferably a chewing gum, a chewable tablet, a tablet, a melt tablet, a lozenge or a hard boiled candy. Of particular interest is a coated chewing gum.

When the below description relates to coated chewing gums or tablets such description should be understood to apply mutatis mutandis also to the other coated oral dosage forms of the present application.

The present invention also provides a method for obtaining reduction of the urge to smoke or use tobacco containing material and/or for providing a sense of smoking satisfaction without smoking, comprising the steps of replacing at least partly the tobacco containing material with above said coated oral dosage form, administering to a subject a coated oral dosage form containing nicotine in any form into the oral cavity of the subject and allowing the nicotine in any form of the coated oral dosage form to be released in the saliva in the oral cavity and absorbed by the subject.

Furthermore, the present invention provides a system for delivering nicotine in any form to a subject, comprising said coated oral dosage form and at least one other means for obtaining reduction of the urge to smoke or use of tobacco as well as a system for obtaining reduction of the urge to smoke or otherwise use of tobacco and/or for providing a sense of smoking satisfaction without smoking, comprising a coated oral dosage form according to above and at least one other method for obtaining, reduction of the urge to smoke or otherwise use of tobacco. Said system may be a system wherein the at least other method is selected from the group consisting of administration through mouth sprays, nasal sprays, transdermal patches, inhaling devices, lozenges, tablets and parenteral methods, subcutaneous methods, and transmucousal methods; or otherwise use of tobacco.

Still furthermore the present invention relates to a coated oral dosage form comprising at least one core, nicotine in any form and/or a nicotine mimicking agent, at least one coating layer and optionally at least one or more other additives, wherein said at least one coating layer is buffered with at least one amino acid.

By using an amino acid as the only buffer, or as the main buffer, in said coated oral dosage form the problems with the gum product according to WO 02/102357, ie off-notes from the buffers used and too long drying times for the coating layers, are solved.

Use of the present coated oral dosage form will according to the invention rapidly deliver nicotine in any form to a subject and will also be used for obtaining a quick and/or sustained and/or complete reduction of the urge to smoke or use tobacco and/or for providing a sense of smoking satisfaction without smoking resembling the sense of smoking satisfaction and reduction of the urge to smoke obtained after regular smoking or use of tobacco.

The core of the captioned coated gum and the captioned non-coated gum have essentially the same composition—except for their respective different content of nicotine.

### DETAILED DESCRIPTION OF THE INVENTION

Definitions

The term “core” is herein intended to mean an entity or a nucleus onto which one or more coating layers is/are applied.

The term “fast reduction of the urge to smoke or use tobacco” is herein intended to mean an initial priming of the subject so as to achieve a reduction of the urge to smoke or use tobacco.

The term “sustained” is herein intended to mean prolonged over time.

The term “complete reduction” or “complete” is herein intended to mean complete or substantially complete reduction.

The term “controlled release” is intended to mean a release of a substance from a gum or tablet by the aid of active chewing or sucking of the gum or tablet in the oral cavity of the subject, whereby the active chewing or sucking is controlling the amount of substance released.

The term “slow release” is intended to mean that the nicotine is released from the gum or tablet upon, eg chewing, over a period of time eg several minutes to an hour.

The term “unit formula” is intended to mean one chewing gum or tablet product.

The term “transient” is intended to mean a non-permanent change, upon which the relevant state, eg biological or physiological state, after a certain period of time will return to its value or behaviour prior to said change.

The terms “buccal” and “buccally” are herein intended to pertain to all of or any part of the tissue of the oral cavity.

The term “intraoral delivery” is herein intended to mean delivery into the systemic blood circulation by means of absorption of the active principle by any tissue of the oral cavity.

The Coated Oral Dosage Form

Presently existing nicotine chewing gums, and other oral dosage forms, provide a slow release and a slow uptake of nicotine compared to smoking. This does not always reliably create the actual sense of satisfaction when smoking, where an initial fast uptake of nicotine is achieved giving the smoker or tobacco user, ie the subject, a sense of satisfaction. Accordingly, as revealed above, the present invention relates to a coated chewing gum or tablet product for improving the absorption of nicotine in a subject, and wherein the absorption is quicker than by using current means and methods known in the art of nicotine chewing gums. Such a rapid transmucosal uptake of the nicotine in the oral cavity is expected to give more of a cigarette like sense of satisfaction and a more rapid reduction of the urge to smoke and use tobacco.

The present coated chewing gum or tablet product comprises at least one core, nicotine in any form and/or a nicotine-mimicking agent, at least one coating layer and at least one other additive, wherein at least one of said coating layer is buffered.

The at least one core may be buffered in different embodiments. The core may be buffered with the same or different ways of buffering as the at least one coating layer.

Said buffering of the at least one coating layer and optionally the at least one core generates a coated chewing gum or tablet product giving improved absorption kinetics of nicotine compared to in the art known chewing gum or tablet products. Most importantly, the buffering is achieved at least partly through use of an amino acid.

The chewing gum or tablet product may be a medicated chewing gum or tablet. Medicated chewing gums are herein intended to mean solid or semi-solid, single-dose preparations with a base consisting mainly of gum that are intended to be chewed but not swallowed, where the chewing gums act as a drug delivery system. They contain one or more active substances, which are released by chewing. In the present invention the active substance is nicotine and/or a nicotine mimicking agent intended for systemic delivery.

The Buffering Agent

Absorption of nicotine from the oral cavity to the systemic circulation is dependent on the pH of the saliva, pH of the blood plasma and the acid-base equilibrium of nicotine, which is about pKa=7.8 at 37° C. Assuming a pH of the saliva of 6.8, only about 10% of the nicotine will be in the non-charged base form. Thus, in order to promote absorption of nicotine in a free base form, which is the form predominantly absorbed through the mucosa, the pH of the saliva must preferably be increased to at least pH 7 and to at most pH 10, more preferably to at least pH 8 and at most pH 9.5. At a pH of 9.0 more than 90% of the nicotine will be in the free readily absorbable base form.

According to the invention, the oral formulation is buffered by use of substances, agents or other means, which at least partly comprise an amino acid, preferably an endogenous amino acid.

As said above many of the amino acid type of buffering agents possess no intrinsic taste. Further, many of the common amino acids, especially the endogenous ones, can be classified as harmless from a toxicity point of view since they are present in large amounts, several grams per day, in common nutrition.

As least some of the below criteria should preferably be used when selecting amino acids useful as buffers in nicotine-containing formulations:

1) pKa in the interval 8.0-9.6 (as the system should buffer in the pH area above nicotine's pKa value at 25° C.).
2) Solubility in water more than around 10 g/kg.
3) Useful from a toxicity point of view.
4) Preferably already used as buffer in pharmaceutical formulations devoid of nicotine.

The most useful amino acids are listed in below Table 1.

TABLE 1
Especially useful amino acids.

| | CAS | pKa value (in | Solubility in |
|---|---|---|---|
| Compound | number | interval 8.0-9.6) | water, g/kg |
| Arginine | 74-79-3 | 9.00 | 182.6^{a)} |
| Aspargine | 70-47-3 | 8.73 | 25.1 |
| Glutamic acid | 56-86-0 | 9.58 | 8.61^{a)b)} |
| Glutamine | 56-85-9 | 9.00 | 42 |
| Glycine | 56-40-6 | 9.58 | 250.9 |
| Histidine | 71-00-1 | 9.09 | 43.5 |
| Isoleucine | 73-32-5 | 9.60 | 34.2 |
| Leucine | 61-90-5 | 9.58 | 22.0 |
| Lysine | 56-97-1 | 9.16 | Very soluble^{a)b)} |
| Methionine | 63-68-3 | 9.08 | 56 |
| Phenylalanine | 63-91-2 | 9.09 | 27.9 |
| Serine | 56-45-1 | 9.05 | 50.2 |
| Threonine | 72-19-5 | 8.96 | 98.1 |
| Valine | 72-18-4 | 9.52 | 88.5 |
| Cysteic acid | 13100-82-8 | 8.70 | Very soluble |
| N-Glycylglycine | 556-50-3 | 8.10 | No information |
| Ornithine | 70-26-8 | 8.78 | Very soluble |

^{a)}reported as buffer in non-nicotine-containing pharmaceutical formulations.
^{b)}low or uncertain value on solubility in water.

The captioned data on the amino acids are taken from “Handbook of Chemistry and Physics”, 85^{th} edition; Table 7-1 (“20 standard amino acids that are the basic constituents of proteins”) and Table 7-2 (“Amino acids and related compounds of biochemical importance”).

The buffering is designed so as to achieve a transient buffering of the saliva of a subject at an elevated pH value during melting, disintegration or dissolution of the oral formulation. As the change is transient, the pH will return to its normal value after a certain period of time.

By employing said increase in pH of the saliva, the transmucousal uptake of nicotine in the oral cavity is increased compared to the nicotine uptake when the saliva is not buffered according to the invention. Also, since the transmucousal uptake of nicotine in the oral cavity according to the invention is faster than for nicotine not being buffered according to the invention, less nicotine will be swallowed to reach the gastrointestinal (G.I.) tract. The nicotine that reaches the G.I. tract will be subjected to first pass metabolism which reduces the total amount of intact nicotine absorbed. This means that the bio-availability of nicotine that is not co-administered with a buffer will generally be lower than when administered together with a buffer.

Thus according to the invention, the coated chewing gum or tablet product is buffered. This may be achieved by including physiologically acceptable buffering substances or agents, or by other means, whereby said substances, agents or other means at least partly comprise an amino acid. Other means include any component in the product, which does not normally act as a buffering agent, such as a self-buffering additive or a gum base.

According to the invention, at least one coating layer is buffered. In specific embodiments, also the at least one core is buffered.

In specific embodiments, the at least one coating layer is buffered in such a way that upon administration of the gum or tablet the pH of the saliva is increased 0.3-4 pH units, preferably 0.5-2 pH units. The buffering is designed so as to achieve a transient buffering of the saliva of a subject during melting, disintegration or dissolution of the coating layer or layers. As the change is transient, the pH will return to its normal value after a certain period of time.

Similarly, the at least one core may be buffered. This may allow said change in the pH to be ensured during chewing of the core or sucking of the gum or tablet product, where the chewing or sucking allows the suitable buffer agent or substance or other means to produce a transient change in the pH of the saliva, eg an increase in the pH.

By employing the change in pH, for example an increase in said pH of the saliva, the transmucosal uptake of nicotine in the oral cavity is changed, eg increased compared to the nicotine uptake when the saliva is not buffered according to the invention. Also, since the transmucosal uptake of nicotine in the oral cavity according to the invention is faster than for nicotine which has not been buffered according to the invention, less nicotine will be swallowed to reach the gastrointestinal (G.I.) tract. The nicotine that reaches the G.I. tract will be subjected to first pass metabolism which reduces the total amount of intact nicotine absorbed. This means that the bio-availability of nicotine that is not co-administered with a buffer according to the invention will generally be lower than when administered together with a buffer as described in this invention.

Further embodiments of the invention include combinations wherein the at least one coating layer is buffered by the use of an amino acid, optionally together with a buffer selected from the group consisting of a carbonate including bicarbonate or sesquicarbonate, phosphate, glycerophosphate or citrate of an alkali metal, such as potassium or sodium, or ammonium, and mixtures thereof.

Still further embodiments may encompass use of an amino acid together with different phosphate systems, such as trisodium phosphate, disodium hydrogen phosphate; and tripotassium phosphate, dipotassium hydrogen phosphate, and calcium hydroxide, sodium glycinate, trometamol; and mixtures thereof.

Alkali metal carbonates and phosphates are preferred additional buffering agents.

In order to increase the buffering capacity still further without correspondingly increasing the pH, one may in specific embodiments use a second or auxiliary buffering agent to the first at least one amino acid buffering agent, such as eg sodium or potassium bicarbonate buffers. The second or auxiliary buffering agent may be selected from the group consisting of alkali metal bicarbonates that are preferred for this purpose. Thus, further embodiments of the invention may comprise an amino acid and a mixture of an alkali metal carbonate or phosphate and alkali metal bicarbonate.

The amount of the buffering agent or agents in the chewing gum or tablet composition is preferably sufficient in the specific embodiments to raise the pH of the saliva to above 7.5, as specified above, to transiently maintain the pH of the saliva in the oral cavity above 7, eg pH 7-10.

The amount of buffer required to achieve said increase in pH of the different administered nicotine forms is readily calculated by the skilled man in the art. The extent and duration of the increase in pH is dependent on type and amount of the buffering agent(s) used as well as where, ie in the at least one coating layer and optionally in the at least one core, the buffer is distributed in the product and is further described within the paragraphs below.

The nicotine may be administered in different forms, eg in different complexes or as a salt.

The Coating

Examples of particular embodiments of the invention include coated gums, tablets or other dosage forms. According to one embodiment of the invention, the chewing gum or tablet is a coated chewing gum or tablet comprising at least one coating layer. The process of coating a chewing gum, a tablet or other oral dosage forms is well known in the art. The present invention provides a coating, to facilitate the uptake of administered nicotine in any form to the subject. Known intentions of coating a chewing gum or tablet product may be to add crispiness, enhance taste, or to protect the gum or tablet, eg during storage, or to tone down bad or irritating tastes of the gum or tablet product.

Particular embodiments according to the invention may use hard coating, film coating, press/compression coating or melt coating.

For the film and hard coating, the coating procedure may be manual or the coating may be sprayed onto the gum or tablet core/pellet in rotating pans of different shapes or fluidised beds in combination with evaporation of the solvent, eg water or organic solvent.

Hard coating is a multistep process and may be divided into the following steps:
1. sealing of the cores
2. subcoating
3. smoothing, or glossing
4. colouring
5. polishing
6. optionally printing

Hard coated cores have a smoother profile with less visible edges remaining from the original core. Sub-coating, by dusting with powder on a sugar alcohol solution or application of dry powder in the sugar alcohol solution, may be used. The core may be hard coated by a panning technique, eg using a hard coating pan, or by other more sophisticated techniques capable of some degree of automation.

The sugar in a hard coating may be selected from the group consisting of sucrose, sugar alcohols, polyalcohols, polyols and mixtures of two or more of the foregoing.

The sugar used in the hard coating may according to specific embodiments also be an artificial sweetener, being (1) low or substantially free of calories and (2) less caries promoting than regular sugar, or a combination with sugar and/or sugar alcohol. Examples of artificial sweeteners and of such combinations are given below under Other additives.

Film coating involves the deposition, usually by a spray method, of a thin film of polymer surrounding the core. The solution may be sprayed to a rotated, mixed bed. The drying conditions permit the removal of the solvent so as to leave a thin deposition of coating material around each core.

The composition of the coating solutions and suspensions may differ during different parts of the process.

Press coating involves the compaction of granular material around an already manufactured core. Using press/compression coating, a further core is pressed on the outside of the initial core/cores.

If nicotine hydrogen tartrate (NHT) is used as the nicotine form then NHT and the buffers are suitably separated from each other in the coating by being kept in separate layers, especially when hard coating is used. A moisture barrier between the NHT-containing layer and the coating comprising the buffer(s) may be applied to prevent interaction between the acid salt NHT and the buffer(s) during the coating process. Suitable moisture barriers are eg apolar lipids and waxes such as carnauba wax, ethyl cellulose or a combination of ethylcellulose and hydroxypropyl methylcellulose (HPMC) and/or plasticizer from an organic solvent or solvent mixture, aqueous ethylcellulose dispersion such as Aquacoat EDC (FMC Corp., Philadelphia, Pa.) or Surelease (Colorcon, West Point, Pa.) preferably in combination with plasticizer, Sepifilm LP 007 or LP 010 (Seppic, Paris, France)—based mainly on HPMC and stearic acid-, Opadry AMB or High Performance Opadry II (Colorcon)—based mainly on polyvinylalcohol-, and polymethacrylates as Eudragit L30 D-55 or EPO (Röhm, Germany). Depending on the type of barrier film selected the moisture barrier preferably accounts for a weight of around 0.3% to around 5% of the total weight of the coating.

One or more additives may be added to the coating or the core/s. Additives are further described in the paragraph Other additives.

The Core

The amount of gum base in a coated chewing gum according to the invention is about 15-80% by weight of the total gum core, and preferably at least about 40%., such as in the range of 40-80%. The amount of gum base employed for the most desirable slow release of nicotine is usually in the higher ranges when nicotine is employed as free base or when an absorbed form is used.

The gum base may be of any conventional nature known in the art. For example it may comprise a gum base of natural or synthetic origin readily available from a commercial source. Natural gum bases include eg chicle, jelutong-, lechi de caspi-, soh-, siak-, katiau-, sorwa-, balata-, pendare-, malaya-, and peach gums, natural cautchouc and natural resins such as dammar and mastix. Synthetic gum bases are a mixture of:
elastomers (for example polymers and masticating substances),
plasticizers (for example resins, elastomers and solvents),
fillers (for example texturizers and water-insoluble adjuvants),
softeners (for example fats),
emulsifiers,
waxes,
antioxidants,
and anti-tacking agents (for example vinyl polymers and hydrophilic resin).

Other examples of gum bases are gums including agar, alginate, arabic gum, carob gum, carrageenan, ghatti gum, guar gum, karaya gum, pectin, tragacanth gum, locust beam gum, gellan gum and xanthan gum.

Examples of gelling agents comprise gum arabic, starch, gelatine, agar, and pectin.

When the nicotine in any form and the buffering agent or agents are incorporated in the chewing gum mass in accordance with the present invention, it is possible to employ a wide variety of chewing gum compositions and amounts of the chewing gum base. Different chewing gum products may be composed depending on the consumer's preference and the purpose of use, in respect of the nicotine level, nicotine distribution and other additives.

The above components may be of qualities suitable for the manufacturing of gums using the mixing, rolling and scoring technology and using the direct compression technology respectively.

As for the core of a tablet, see Example 6.

The Active Ingredient

According to the invention, the coated chewing gum or tablet product comprises nicotine in any form and/or a nicotine mimicking agent. In specific embodiments, the nicotine is part of the at least one coating layer or, if multiple layers are used. at least one of the at least one coating layers.

In still further embodiments, the nicotine is a part of the chewing gum or tablet core or, if multiple cores are used, at least one of the chewing gum or tablet cores.

In still even further embodiments, the nicotine is part of the at least one coating layer or at least one of the at least one coating layers and the chewing gum or tablet core or at least one of the chewing gum or tablet cores to give a fast transmucosal uptake of the nicotine in the oral cavity of a subject so as to obtain a rapid kick or reduction of the urge to smoke and/or use tobacco. Thereby may also be achieved a systemic maintenance level of nicotine.

With nicotine it is intended to include nicotine, 3-(1-methyl-2-pyrrolidinyl)-pyridine, with its base form, including synthetic nicotine as well as nicotine extracts from tobacco plants, or parts thereof, such as the genus Nicotiana alone or in combination, or pharmaceutically acceptable salts.

The nicotine compound should ultimately be in a saliva soluble form to facilitate the rapid release of the nicotine agent into the saliva in the oral cavity and, further, the subsequent uptake of the nicotine from the saliva in the oral cavity into the systemic circulation of the subject.

Nicotine may be used in the form of nicotine resinate complex, NRC. The release of nicotine from NRC is increased in the presence of a buffer.

In preferred embodiments, the nicotine in any form is selected from the group consisting of a nicotine salt, the free base form of nicotine, a nicotine derivative, such as a nicotine cation exchanger, a nicotine inclusion complex (for example nicotine in complex with betacyclodextrin) or nicotine in any non-covalent binding; nicotine bound to zeolites; nicotine bound to cellulose or starch micro-spheres; and mixtures of nay of the foregoing.

Numerous nicotine salts are known, and may be used, eg the salts presented in below Table 2, such as preferably the monotartrate, hydrogen tartrate (also called bi-tartrate), citrate, malate, and/or hydrochloride.

TABLE 2
Possible acids used for nicotine salt formation

| Acid | Molar ratio* of acid:nicotine |
|---|---|
| Formic | 2:1 |
| Acetic | 3:1 |
| Propionic | 3:1 |
| Butyric | 3:1 |
| 2-Methylbutyric | 3:1 |
| 3-Methylbutyric | 3:1 |
| Valeric | 3:1 |
| Lauric | 3:1 |
| Palmitic | 3:1 |
| Tartaric | 2:1 |
| Citric | 2:1 |
| Malic | 2:1 |
| Oxalic | 2:1 |
| Benzoic | 1:1 |
| Gentisic | 1:1 |
| Gallic | 1:1 |
| Phenylacetic | 3:1 |
| Salicylic | 1:1 |
| Phthalic | 1:1 |
| Picric | 2:1 |
| Sulfosalicylic | 1:1 |
| Tannic | 1:5 |
| Pectic | 1:3 |
| Alginic | 1:2 |
| Hydrochloric | 2:1 |
| Chloroplatinic | 1:1 |
| Silicotungstic | 1:1 |
| Pyruvic | 2:1 |
| Glutamic | 1:1 |
| Aspartic | 1:1 |

*recommended level at production

The inclusion complex may be a nicotine-cyclodextrin (1-1) compound, such as nicotine-β-cyclodextrin.

Suitable cation exchangers are given in below Table 3 and are further disclosed in U.S. Pat. No. 3,845,217. Preferred are nicotine cation exchangers of polyacrylates, such as the Amberlite collection from Rohm & Haas.

TABLE 3
Representative cation exchangers

| Name | Type of crosslinked polymer | Manufacturer |
|---|---|---|
| Amberlite IRC 50 | Divinylbenzene-methacrylic acid | Rohm & Haas |
| Amberlite IRP 64 | Divinylbenzene-methacrylic acid | Rohm & Haas |
| Amberlite IRP 64M | Divinylbenzene-methacrylic acid | Rohm & Haas |
| BIO-REX 70 | Divinylbenzene-acrylic acid | BIO-RAD Lab. |
| Amberlite IR 118 | Styrene-divinylbenzene | Rohm & Haas |
| Amberlite IRP 69 | Styrene-divinylbenzene | Rohm & Haas |
| Amberlite IRP 69M | Styrene-divinylbenzene | Rohm & Haas |
| BIO-REX 40 | Phenolic | BIO-RAD Lab. |
| Amberlite IR 120 | Styrene-divinylbenzene | Rohm & Haas |
| Dowex 50 | Styrene-divinylbenzene | Dow Chemical |
| Dowex 50W | Styrene-divinylbenzene | Dow Chemical |
| Duolite C 25 | Styrene-divinylbenzene | Chemical Process Co |
| Lewatit S 100 | Styrene-divinylbenzene | Farbenfabriken Bayer |
| Ionac C 240 | Styrene-divinylbenzene | Ionac Chem. |
| Wofatit KP S 200 | Styrene-divinylbenzene | I. G. Farben Wolfen |
| Amberlyst 15 | Styrene-divinylbenzene | Rohm & Haas |
| Duolite C-3 | Phenolic | Chemical Process |
| Duolite C-10 | Phenolic | Chemical Process |
| Lewatit KS | Phenolic | Farbenfabriken Bayer. |
| Zerolit 215 | Phenolic | The Permutit Co. |
| Duolite ES-62 | Styrene-divinylbenzene | Chemical Process |
| BIO-REX 63 | Styrene-divinylbenzene | BIO-RAD Lab. |
| Duolite ES-63 | Styrene-divinylbenzene | Chemical Process |
| Duolite ES-65 | Phenolic | Chemical Process |
| Ohelex 100 | Styrene-divinylbenzene | BIO-RAD Lab. |
| Dow Chelating Resin A-1 | Styrene-divinylbenzene | Dow Chemical Company |
| CM Sephadex C-25 | Dextran | Pharmacia Fine Chemicals |
| SE Sephadex C-25 | Dextran | Pharmacia Fine Chemicals |

The product according to the invention may also comprise a nicotine mimicking agent. Such an agent may be any suitable agent with a nicotine-like acrid burning taste providing a tingling sensation in the mouth and in the throat. Examples of nicotine mimicking agents are capsaicin, piperine and zingerone.

One or more additives may be added to the coating or the core/s. Additives are further described in the below paragraph Other additives.

Amount and Distribution of the Nicotine

The nicotine in any form according to the invention is formulated to provide the subject with a dose to achieve an effect. The effect may be to provide a sense of smoking satisfaction without smoking. Another effect of the administered nicotine in any form may be a reduction of the urge to smoke or use tobacco.

The effect may also be a combination of reduction of the urge to smoke and smoking satisfaction without smoking. The amount of the nicotine should be sufficient to provide such an effect in a subject. This amount may, of course, vary from person to person.

According to the invention, embodiments of the chewable gum or tablet product comprise embodiments wherein nicotine in any form is present in an amount of 0.05-10 mg calculated as the free base form of nicotine per piece coated chewing gum or tablet product. This may in different embodiments include 0.05, 0.5, 1, 2, 3, 4, 5, 6, 7, 8, 9, or 10 mg calculated as the free base form of nicotine per piece coated chewing gum or tablet product.

Still preferred embodiments may contain embodiments where the nicotine in any form is present in an amount of 0.5-6 mg calculated as the free base form of nicotine per piece coated chewing gum or tablet product.

Even more preferred embodiments contain the nicotine in any form in an amount of 0.5-4 mg calculated as the free base form of nicotine per piece coated chewing gum or tablet product.

According to certain embodiments of the invention, the nicotine in any form is part of the at least one coating layer or at least one of the at least one coating layer.

The nicotine in any form may be in an amount of 0-8 mg calculated as free base form in at least one of the at least one coating layer. Still further embodiments comprise nicotine in an amount of 0.1-6 mg in at least one of the at least one coating layers, or even more preferably, in an amount of 0.1-5 mg in at least one of the at least one coating layer.

The nicotine in any form may be distributed in the core and/or different coating layers in different embodiments. Different distributions of the nicotine throughout the coated chewing gum or tablet will imply administration of the nicotine to the subject in different ways. This may, then, provide several possibilities to adjust the composition of the coated chewing gum or tablet according to different needs of different subjects depending on the urge to smoke or use tobacco of the subject.

Release and Uptake of Nicotine

Currently available nicotine-containing formulations for intraoral uptake, such as chewing gums and tablets, provide a slow release and a slow uptake of nicotine compared to smoking.

The release of the nicotine in the coated pharmaceutical formulation according to the invention proceeds in at least one step as follows.

I) The dissolution of the one or more buffering agents in the coating, and optionally in the core(s), provides for optimized adjustment of the pH of the liquid in the oral cavity.

II) If the nicotine is, as in preferred embodiments, in a defined amount, such as the amounts described above according to different embodiments, in at least one of the at least one coating layers defined above the release of the nicotine takes place when the coating of the coated chewing gum or tablet is allowed to melt, disintegrate or dissolve to expose the chewable gum or tablet core in said product. The nicotine and its various forms is released from the coating into the saliva in the oral cavity during the time period when the coating is allowed to melt, disintegrate or dissolve such as with the use of a chewable or suckable gum or tablet. The nicotine in any form may then further be absorbed by the subject.

III) The nicotine in any form from the chewable or suckable gum or tablet is released by controlled release, eg by chewing or sucking the gum or tablet core whereby the chewing is controlling the amount of released nicotine from the gum or tablet core. The release of the nicotine is thereby sustained over a period of time. This period of time may be, in different embodiments about 5, 10, 20, 30 or 40 minutes.

The release may be varied by the incorporation of the nicotine in any form in a given quantity into the coating layers and/or the gum or tablet core.

Not only the amount of the nicotine released from the different parts of the chewing gum or tablet product is of value, but also, according to the present invention the specific transmucosal uptake from the oral cavity of the nicotine to the systemic circulation of the subject whereby the one or more buffering agents account for provision of a suitable adjustment of the pH of the liquid of the oral cavity.

According to the present invention a sense of satisfaction may be reached after a short period of time due to a rapid initial burst dose of nicotine in the coating followed by a rapid transmucosal uptake in the oral cavity due to the buffered coating. The intraoral uptake of nicotine from the present coated pharmaceutical formulation is preferably more rapid than from non-coated solid or semisolid pharmaceutical formulations for intraoral uptake with the same total nicotine content.

Other Additives

Other additives may be added optionally to the core and/or to coating layers.

Optional additives comprise at least one or more additive selected from the group consisting of stabilisers, such as preservatives, eg antioxidants; softeners, thickening agents, filling agents, film forming agents, emulsifiers, glidants, lubricants, sweeteners, flavours, aromatics, enhancers, colouring agents, vitamins, minerals, fluorine, breath fresheners and tooth whitening agents and mixtures thereof. According to the invention, at least one of such additives is optionally added to the product.

Enhancers are added essentially to improve, ie increase, the transmucosal uptake from the oral cavity.

Sweeteners are added essentially to improve the taste. Sweeteners comprise one or more members selected from synthetic or natural sugars (for example any form of carbohydrates suitable for use as a sweetener), as well as so called artificial sweeteners such as saccarin, sodium saccarin, aspartame (sold as NutraSweet®), acesulfame K or acesulfame, potassium acesulfame, thaumatin, glycyrrhizin, sucralose, dihydrochalcone, alitame, miraculin, monellin, stevside.

Suitable sweeteners may be selected from the group consisting of sugar alcohols, such as sorbitol and xylitol, single sugars including sugars extracted from sugar cane and sugar beet (sucrose), dextrose (also called glucose), fructose (also called leavulose), and lactose (also called milk sugar); sorbitol, mannitol, glycerol, xylitol, erythritol, maltitol syrup (or hydrogenated starch hydrolyzate), isomalt, lactitol; and mixtures of sugars including glucose syrup, (for example starch hydrolysates, containing a mixture of dextrose, maltose and a range of complex sugars), invert sugar syrup (for example sucrose inverted by invertase (also called sucrase or sacchrase) containing a mixture of dextrose and fructose), high sugar content syrups (such as treacle and honey containing a mixture of particular leavulose, dextrose, maltose, lactitole, sucrose, resins, dextrin and higher sugars); and malt or malt extracts.

The flavour and aroma additives may comprise one or more synthetic or natural flavouring or aromatizing agents.

Flavour and aroma agents may be selected from essential oils including distillations, solvent extractions, or cold expressions of chopped flowers, leaves, peel or pulped whole fruit comprising mixtures of alcohols, esters, aldehydes and lactones; essences including either diluted solutions of essential oils, or mixtures of synthetic chemicals blended to match the natural flavour of the fruit, eg strawberry, raspberry and black currant; artificial and natural flavours of brews and liquors, eg cognac, whisky, rum, gin, sherry, port, and wine; tobacco, coffee, tea, cocoa, and mint; fruit juices including expelled juice from washed, scrubbed fruits such as lemon, orange, and lime; spear mint, pepper mint, wintergreen, cinnamon, cacoe/cocoa, vanilla, liquorice, menthol, eucalyptus, aniseeds, nuts (e g peanuts, coconuts, hazelnuts, chestnuts, walnuts, colanuts), almonds, raisins; and powder, flour, or vegetable material parts including tobacco plant parts, eg genus Nicotiana, in amounts not contributing significantly to the level of nicotine, and ginger.

Colouring additives may be selected from dyes being approved as a food additive.

Stabilizing additives may be selected from the group consisting of antioxidants including vitamin E, ie tocopherole, ascorbic acid, sodium pyrosulfite, butylhydroxytoluene, butylated hydroxyanisole, edetic acid and edetate salts; and preservatives including citric acid, tartaric acid, lactic acid, malic acid, acetic acid, benzoic acid, and sorbic acid. Preferred embodiments comprise an antioxidant as the stabiliser, and even more preferably the antioxidant vitamin E and/or butylated hydroxytoluene (BHT).

Method for Delivering Nicotine in any Form to a Subject

According to the invention, a method for delivering nicotine in any form to a subject comprises the steps of

a) administering to a subject a coated chewing gum or tablet product containing nicotine in any form according to the invention into the oral cavity of the subject, and

b) allowing the nicotine in any form in the coated chewing gum or tablet product to be released in the saliva in the oral cavity and absorbed into the blood plasma of the subject.

According to the invention, the transmucosal uptake of the nicotine in the oral cavity is more rapid than with presently known oral pharmaceutical formulations.

The method for delivering nicotine in any form may further comprise the step of

c) administering the nicotine in any form in a sustained way over a period of time to the subject, for example at least 5, 10, 20, 30 or 40 minutes.

Method for Obtaining Reduction of the Urge to Smoke or Use of Tobacco

A method for obtaining reduction of the urge to smoke or use tobacco-containing material and/or for providing a sense of smoking satisfaction without smoking according to the invention comprises the steps of

a) replacing at least partly the nicotine-containing tobacco products with a coated oral dosage form according to the present invention,

b) administering to a subject a coated oral dosage form containing nicotine in any form according to the present invention into the oral cavity of the subject, and

c) allowing the nicotine in any form in the coating of the coated oral dosage form to be released into the saliva in the oral cavity and absorbed by the subject.

Further embodiments of the method for delivering nicotine to a subject may comprise the steps of combining at least one other method for obtaining reduction of the urge to smoke or use of tobacco with the product of the invention.

Tobacco containing material may be material used for eg smoking, snuffing or chewing and may comprise a cigarette, a cigar, pipe tobacco, snuff, snus and chewing tobacco.

The coated oral dosage form may be used for obtaining a quick and/or sustained and/or complete reduction of the urge to smoke or use of tobacco and/or for providing a sense of smoking satisfaction without smoking as further discussed below.

The fast relief provides the subject with a sense of rapid smoking satisfaction without smoking. Such a satisfaction will decrease the craving more rapidly than other known solid or semisolid oral dosage forms.

The quick craving relief is obtained when a dosage of nicotine is released from at least one of the at least one coating layers of the coated oral dosage form in embodiments wherein nicotine is in the coating layers in the presence of one or more buffering agents in the coating and optionally in the core(s). This provides the subject with an initial rapid transmucosal uptake of nicotine in the oral cavity that will induce an initial peak, which results in that the subject gets a feeling or sense of satisfaction and the initial craving will disappear.

Sustained Reduction of the Urge to Smoke or Use of Tobacco

The invention may provide sustained reduction of the urge to smoke or use tobacco and give the subject an ability to feel a sense of satisfaction even after the initial craving relief. A sustained craving relief is obtained by chewing or sucking the core part of the coated oral dosage to allow a sustained uptake of the nicotine. The sustained craving relief and/or feeling or sense of satisfaction of the subject will continue as long as the subject maintains the blood plasma levels of nicotine at a level high enough to reach this sense of feeling.

The subject may achieve this sustained relief by chewing the core of the coated oral dosage form over a period of time, such as 5, 10, 20, 30 or 40 minutes or longer, thereby obtaining the slow release by chewing.

Cessation of the Urge to Smoke or Use of Tobacco

For some of the users, it may be a goal to terminate the usage of nicotine completely, due to several reasons eg health, economical, social or behavioural. This may be achieved by further decreasing the amount of nicotine in any form gradually over time. In a specific embodiment of the invention, the method described above for obtaining craving relief may further comprise the steps of decreasing the amount of nicotine in the total coated oral dosage form product described above gradually over time, so as to achieve a complete relief of tobacco craving. This method results in a weaning process gradually over time.

Different types of smokers reach the sense of reduced craving at different plasma levels of nicotine. This may, of course, affect the individual types of administration programs of a coated chewing gum or tablet according to the invention. Different types of smokers include eg peak seekers or smokers that crave a plasma level of nicotine, which is constantly above the level for withdrawal symptoms.

One strategy may be to lower the frequency of the administered coated oral dosage form. Other embodiments include varying the dose of the nicotine in said coated oral dosage forms as well as the combination of these two. Also, the strategy may include a coated oral dosage form with substantially no nicotine in any form. Such a coated oral dosage form may be administered at the end of the treatment period, when the craving is low or substantially absent.

Systems for Delivering Nicotine and for Obtaining Craving Relief

According to the invention there is a system for delivering nicotine in any form to a subject. Such a system comprises a coated oral dosage form according to the invention and at least one other means for obtaining reduction of the urge to smoke.

Another system according to the invention may also be a system for obtaining reduction of the urge to smoke or use of tobacco and/or for providing a sense of smoking satisfaction without smoking. Such a system comprises a coated oral dosage form according to the invention and at least one other method for obtaining reduction of the urge to smoke or use tobacco. Other methods may also be a concomitant or concurrent method selected from the group consisting of administration through mouth sprays, nasal sprays, transdermal patches, inhaling devices, lozenges, tablets and parenteral methods, subcutaneous methods, and transmucosal methods; or use of tobacco.

In a specific embodiment, the at least other method comprises administration of nicotine.

Use of the Coated Oral Dosage Form

The use of the coated oral dosage form according to the invention is for obtaining a fast and/or sustained and/or complete reduction of the urge to smoke and use tobacco or for providing a sense of smoking without smoking as described above.

The dose of the nicotine is chosen to give the subject an individual sensory perception and satisfaction with an effect of the nicotine in any form. The use of the coated oral dosage form may also be a sole use according to the invention or a combination with other means or methods known in the field of drug abuse. Specifically, the present invention may be used in combination with other means as described above in the methods in the paragraphs above.

According to the invention, a use of a coated oral dosage form according to the invention is also disclosed for delivering nicotine in any form to a subject.

Production of the Coated Oral Dosage Form

Coated oral dosage forms according to the invention can be maintained in several production steps depending on the total number of cores and the total number of coated layers to be included.

One method for the production of the coated oral dosage form according to the invention is disclosed below. Alternatively other production methods would be useful, eg manufacturing using compression technology.

The method comprises the steps of

Example 6B Wet granulated nicotine chewable tablet (600 mg core weight)

| | 0 mg | | 1 mg | | | |
|---|---|---|---|---|---|---|
| | Unit | 0.5 mg | Unit | 2 mg | 3 mg | 4 mg |
| | for- | Unit | for- | Unit | Unit | Unit |
| | mula | formula | mula | formula | formula | formula |
| | (mg) | (mg) | (mg) | (mg) | (mg) | (mg) |
| Active ingredients | | | | | | |
| Nicotine hydrogen | 0 | 1.7 | 3.4 | 6.8 | 10.2 | 13.6 |
| tartrate | | | | | | |
| Other ingredients | | | | | | |
| Dextrose | 590 | 588 | 585 | 584 | 575 | 570 |
| PVP | 4 | 4 | 4 | 4 | 4 | 4 |
| PEG 6000 | 6 | 6 | 6 | 6 | 6 | 6 |
| Water | q.s. | q.s. | q.s. | q.s. | q.s. | q.s. |

### Manufacturing Method:

Nicotine hydrogen tartrate and dextrose powders are dry-blended and then granulated with a solution of PVP in water in a fluid bed granulator. The granulated material is then sieved, dry-blended with PEG and compressed into tablets. The cores are then coated using any of the methods according to Examples 1-4.

a) providing at least one core, and/or providing at least one nicotine containing core,

b) providing nicotine in any form,

c) providing at least one coating layer that is buffered with at least one amino acid,

d) adding the nicotine in any form to the at least one core and/or to the at least one coating, and

e) coating the at least one core with the at least one coating layer that is buffered.

The method may in specific embodiments further comprise

f) buffering the at least one core, and/or

g) providing at least one coating layer not being buffered, and optionally

h) adding the nicotine in any form to at least one of said at least one coating layer not being buffered, and optionally

i) providing the nicotine in the coating and the buffer in the coating in separate layers, preferably separated by a moisture barrier.

In one embodiment, the nicotine is selected from the group consisting of a nicotine salt, the free base form of nicotine, a nicotine derivative, such as a nicotine cation exchanger, a nicotine inclusion complex or nicotine in any non-covalent binding; nicotine bound to zeolites; nicotine bound to cellulose or starch micro-spheres; and mixtures thereof.

The at least one coating layer may in some embodiments be buffered by the use of a buffer selected from the group consisting of at least one amino acid or at least one amino acid in combination with a buffer selected from a carbonate buffer, such as the carbonate, bicarbonate, sesquicarbonate of an alkali metal, eg potassium, sodium; or ammonium; sodium glycinate, alkali metal phosphate, sodium or potassium glycerophosphate, trisodium or tripotassium citrate; or trometamol, and mixtures thereof wherein the at least one coating layer is buffered in such a way that upon administration of the gum the pH of the saliva is increased by 0.3-4 pH units. The buffering may be transient.

In still further embodiments, the at least one coating layer is buffered in such a way that upon administration of the gum the pH of the saliva is increased by 0.5-2 pH units.

In the case of chewing gums the core composition may be formed simply by mixing, rolling and scoring or compression of the gum base with at least one of the forms of nicotine, eg the nicotine-ion exchanger complex, or the nicotine as a free base or a salt. Before adding any solid component, except for the gum base, it is desirable to grind and size the solid component first, to ensure good distribution. The mixing is preferably conducted at a suitably elevated temperature depending on the viscosity of the gum core used. The increase in temperature decreases the viscosity of the gum and thereby enables the nicotine and other additives to be evenly and intimately distributed within the core/pellet of the chewing gum. The gum mass with additives is cooled, rolled, scored and hardened sufficiently, and then coated according to the above paragraph The coating and Examples 1-4.

According to the method disclosed in the invention, some embodiments are disclosed where the coating of the at least one chewing gum or tablet core with at least one layer of the at least one buffered coating comprises the steps of

a) film coating, and/or

b) press coating, and/or

c) hard coating, and/or

d) melt coating.

The product may then be analysed and further wrapped according to methods known in the art.

The different embodiments of the invention are manufactured using technology known in the art.

Use for Therapy and Treatment

The coated chewing gum or tablet product according to the invention may be used in therapy. Said therapy may be a treatment of a disease selected from the group consisting of tobacco or nicotine dependence, Alzheimer's disease, Crohn's disease, Parkinson's disease, Tourette's syndrome, ulcerous colitis and post-smoking-cessation weight control.

The nicotine may also be used for the production of a chewing gum or tablet product according to the invention for the treatment of a disease selected from the group consisting of Alzheimer's disease, Crohn's disease, Parkinson's disease, Tourette's syndrome, ulcerous colitis and post-smoking-cessation weight control.

Also disclosed is the use of a coated chewing gum or tablet product for the production of a nicotine-containing chewing gum or tablet product according to the invention for the treatment of a disease selected from the group consisting of tobacco or nicotine dependence, Alzheimer's disease, Crohn's disease, Parkinson's disease, Tourette's syndrome, and ulcerous colitis.

Analysis of Nicotine

The analysis of nicotine uptake and affect according to the invention may be done according to standard procedures known in the art, eg using a bioanalysis for the determination of nicotine or its metabolites in the plasma of a subject.

### EXAMPLES

The below examples are illustrative and non-limiting. Examples 1-4 describe four different coatings and coating compositions that may be used according to the invention, ie hard coating in Example 1, film coating in Example 2, press coating in Example 3 and melt coating in Example 4, all onto a chewing gum or tablet core. The coating is buffered in each case and contains nicotine as well. The coatings in Examples 1-4 may be combined with different cores. Examples of cores are given in Example 5 and further described below.

The skilled person may on the basis of the following examples envisage also other embodiments of the present invention.

Batch sizes for the manufacture of the below formulations may be modified according to the actual need and to the actual production facilities.

### Example 1

### Buffered Hard Coating

Objective

The objective of this example is to provide a hard nicotine-containing and buffered coating. The nicotine is in the amount of 0.5, 1, 2, 3 or 4 mg, respectively.

Material Hard Coating*

A. Nicotine free base as active

| | 0.5 mg | 1 mg | 2 mg | 3 mg | 4 mg |
|---|---|---|---|---|---|
| | unit | unit | unit | unit | Unit |
| | formula | formula | formula | formula | formula |
| Component | (mg) | (mg) | (mg) | (mg) | (mg) |
| Sorbitol | 88.7 | 79.7 | 61.5 | 42.1 | 25.0 |
| Mannitol | 29.1 | 28.8 | 28.4 | 27.2 | 20.7 |
| Xylitol | 160 | 158 | 154 | 151 | 147 |
| Water | q.s.** | q.s.** | q.s. | q.s. | q.s. |
| Gelatin | 3.4 | 3.4 | 3.4 | 3.4 | 3.4 |
| Titanium | 2.5 | 2.5 | 2.5 | 2.5 | 2.5 |
| dioxide | | | | | |
| L-Arginine | 10.8 | 21.6 | 43.2 | 64.8 | 86.4 |
| Nicotine | 0.5 | 1 | 2 | 3 | 4 |
| free base | | | | | |

*hard coating in this example denotes sugar alcohols, not saccharose-based sugar.
**q.s. = quantum satis.

B. Nicotine hydrogen tartrate as active

| | 0.5 mg | 1 mg | 2 mg | 3 mg | 4 mg |
|---|---|---|---|---|---|
| | unit | unit | unit | unit | Unit |
| | formula | formula | formula | formula | formula |
| Component | (mg) | (mg) | (mg) | (mg) | (mg) |
| Sorbitol | 87.2 | 77.3 | 56.8 | 36.0 | 15.7 |
| Mannitol | 29.4 | 28.8 | 28.0 | 27.1 | 26.4 |
| Xylitol | 160 | 158 | 154 | 151 | 147 |
| Water | q.s. | q.s. | q.s. | q.s. | q.s. |
| Gelatin | 3.4 | 3.4 | 3.4 | 3.4 | 3.4 |
| L-Arginine | 10.8 | 21.6 | 43.2 | 64.8 | 86.4 |
| Titanium | 2.5 | 2.5 | 2.5 | 2.5 | 2.5 |
| dioxide | | | | | |
| Nicotine | 1.7 | 3.4 | 6.8 | 10.2 | 13.6 |
| hydrogen | | | | | |
| tartrate | | | | | |
| (corresponding | (0.5) | (1) | (2) | (3) | (4) |
| to | | | | | |
| nicotine free | | | | | |
| base) | | | | | |

### Example 2

### Buffered Film Coating

Objective

The objective of this example is to provide a nicotine-containing and buffered film coating. The nicotine is in the amount of 0.5, 1, 2, 3 or 4 mg, respectively.

Material Film Coating

A. Nicotine free base as active

| | 0.5 mg | 1 mg | 2 mg | 3 mg | 4 mg |
|---|---|---|---|---|---|
| | unit | unit | unit | unit | unit |
| | formula | formula | formula | formula | formula |
| Component | (mg) | (mg) | (mg) | (mg) | (mg) |
| HPMC^{a} | 5 | 10 | 20 | 30 | 40 |
| PEG^{b} | 4.8 | 4.8 | 4.8 | 4.8 | 4.8 |
| Paraffin wax | 0.7 | 0.7 | 0.7 | 0.7 | 0.7 |
| L-Arginine | 10.8 | 21.6 | 43.2 | 64.8 | 86.4 |
| Nicotine free base | 0.5 | 1 | 2 | 4 | 4 |
| Water | q.s. | q.s. | q.s. | q.s. | q.s. |
| Ethanol | q.s. | q.s. | q.s. | q.s. | q.s. |

B. Nicotine hydrogen tartrate as active

| | 0.5 mg | | 1 mg | | 2 mg | | 3 mg | | 4 mg | |
|---|---|---|---|---|---|---|---|---|---|---|
| | unit | | unit | | unit | | unit | | unit | |
| | formula | | formula | | formula | | formula | | formula | |
| Component | (mg) | | (mg) | | (mg) | | (mg) | | (mg) | |
| HPMC^{a} | 5 | 5 | 10 | 10 | 20 | 20 | 30 | 30 | 40 | 40 |
| PEG^{b} | 0 | 4.8 | 0 | 4.8 | 0 | 4.8 | 0 | 4.8 | 0 | 4.8 |
| NHT, | 3.4 | 3.4 | 6.2 | 6.2 | 6.2 | 6.2 | 12.3 | 12.3 | 12.3 | 12.3 |
| (corresponding to | 0.5 | 0.5 | 1 | 1 | 2 | 2 | 3 | 3 | 4 | 4 |
| nicotine free | | | | | | | | | | |
| base) | | | | | | | | | | |
| Paraffin wax | 0.7 | 0.7 | 0.7 | 0.7 | 0.7 | 0.7 | 0.7 | 0.7 | 0.7 | 0.7 |
| L-Arginine | 10.8 | 10.8 | 21.6 | 21.6 | 43.2 | 43.2 | 64.8 | 86.4 | 86.4 | 96.4 |
| Water | q.s. | q.s. | q.s. | q.s. | q.s. | q.s. | q.s. | q.s. | q.s. | q.s. |
| Ethanol | q.s. | q.s. | q.s. | q.s. | q.s. | q.s. | q.s. | q.s. | q.s. | q.s. |

^{a}HPMC = hydroxypropyl methylcellulose
^{b}PEG = polyethylene glycol

### Example 3

### Buffered Press Coating

Objective

The objective of this example is to provide a nicotine-containing and buffered press coating. The nicotine is in the amount of 0.5, 1, 2, 3 or 4 mg, respectively.

Material Press Coating

A. Nicotine hydrogen tartrate as active

| | 0.5 mg | 1 mg | 2 mg | | |
|---|---|---|---|---|---|
| | unit | unit | unit | 3 mg unit | 4 mg unit |
| | formula | formula | formula | formula | formula |
| Component | (mg) | (mg) | (mg) | (mg) | (mg) |
| Xylitol | 740.2 | 727.4 | 702.8 | 677.2 | 652.6 |
| HPMC | 238 | 238 | 238 | 238 | 238 |
| L-Arginine | 10.8 | 21.6 | 43.2 | 64.8 | 86.4 |
| Magnesium stearate | 10 | 10 | 10 | 10 | 10 |
| NHT | 1.7 | 3.4 | 6.8 | 10.2 | 13.6 |
| (corresponding to | 0.5 | 1 | 2 | 3 | 4 |
| nicotine free base) | | | | | |

B. Nicotine resin complex (NRC) or nicotine beta-cyclodextrin complex
(NCC) as active

| | 0.5 mg | | 1 mg | | 2 mg | | 3 mg | | 4 mg | |
|---|---|---|---|---|---|---|---|---|---|---|
| | unit | | unit | | unit | | unit | | unit | |
| | formula | | formula | | formula | | formula | | formula | |
| | (mg) | | (mg) | | (mg) | | (mg) | | (mg) | |
| | NRC | NCC | NRC | NCC | NRC | NCC | NRC | NCC | NRC | NCC |
| Component | (mg) | (mg) | (mg) | (mg) | (mg) | (mg) | (mg) | (mg) | (mg) | (mg) |
| Xylitol | 739 | 737 | 725 | 721 | 699 | 692 | 632 | 662 | 646 | 632 |
| HPMC | 238 | 238 | 238 | 238 | 238 | 238 | 238 | 238 | 238 | 238 |
| L-Arginine | 10.8 | 10.8 | 21.6 | 21.6 | 43.2 | 43.2 | 64.8 | 64.8 | 86.4 | 86.4 |
| Magnesium | 10 | 10 | 10 | 10 | 10 | 10 | 10 | 10 | 10 | 10 |
| stearate | | | | | | | | | | |
| NRC | 2.5 | — | 5 | — | 10 | — | 15 | — | 20 | — |
| (corresponding | 0.5 | — | 1 | — | 2 | — | 3 | — | 4 | — |
| to nicotine free | | | | | | | | | | |
| base) | | | | | | | | | | |
| NCC | — | 4.3 | — | 8.6 | — | 17.1 | — | 25.7 | — | 34.2 |
| (corresponding | — | 0.5 | — | 1 | — | 2 | — | 3 | — | 4 |
| to nicotine free | | | | | | | | | | |
| base) | | | | | | | | | | |

### Example 4

### Buffered Melt Coating

Objective

The objective of this example is to provide a nicotine-containing and buffered melt coating, The nicotine is in the amount of 0, 5, 1, 2, 3 or 4 mg, respectively.

Material Melt Coating

A. Nicotine free base as active

| | 0.5 mg | 1 mg | | | |
|---|---|---|---|---|---|
| | unit | unit | 2 mg unit | 3 mg unit | 4 mg unit |
| | formula | formula | formula | formula | formula |
| Component | (mg) | (mg) | (mg) | (mg) | (mg) |
| Hydrogenated | 176 | 176 | 176 | 176 | 176 |
| vegetable oil | | | | | |
| Cocoa powder | 192 | 198 | 197 | 192 | 192 |
| Aspartame | 2.4 | 2.4 | 2.4 | 2.4 | 2.4 |
| L-Arginine | 10.8 | 21.6 | 43.2 | 64.8 | 86.4 |
| Lecithin | 4 | 4 | 4 | 4 | 4 |
| Nicotine free base | 0.5 | 1 | 2 | 3 | 4 |

B. Nicotine hydrogen tartrate as active

| | 0.5 mg | 1 mg | 2 mg | | |
|---|---|---|---|---|---|
| | unit | unit | unit | 3 mg unit | 4 mg unit |
| | formula | formula | formula | formula | formula |
| Component | (mg) | (mg) | (mg) | (mg) | (mg) |
| Hydrogenated | 176 | 176 | 176 | 176 | 176 |
| vegetable oil | | | | | |
| Cocoa powder | 198 | 198 | 197 | 197 | 192 |
| Aspartame | 2.4 | 2.4 | 2.4 | 2.4 | 2.4 |
| L-Arginine | 10.8 | 21.6 | 43.2 | 64.8 | 86.4 |
| Lecithin | 4 | 4 | 4 | 4 | 4 |
| NHT | 1.7 | 3.4 | 6.8 | 10.2 | 13.6 |
| (corresponding to | 0.5 | 1 | 2 | 3 | 4 |
| nicotine base, mg) | | | | | |

### Example 5

### Gum Cores

Objective

The objective of this example is to provide a core suitable for a chewing gum product according to the invention. The nicotine is incorporated as the free base (NFB), nicotine β-cyclodextrin complex (NCC), nicotine hydrogen tartrate (NHT) or as a nicotine resin complex (NRC). The amount of nicotine in each formula unit, ie per core, is 0, 0.5, 1, 2, 3 or 4 mg.

Principle

The gum core is formed by a mixing, rolling and scoring process or by a compression process.

Composition of the Cores

A. Manufactured by tablet compression process.

| | 0 mg | | 1 mg | 2 mg | | |
|---|---|---|---|---|---|---|
| | Unit | 0.5 mg | Unit | Unit | 3 mg | 4 mg |
| | for- | Unit | for- | for- | Unit | Unit |
| | mula | formula | mula | mula | formula | formula |
| | (mg) | (mg) | (mg) | (mg) | (mg) | (mg) |
| Active ingredient | | | | | | |
| Nicotine resin | 0 | 2.5 | 5 | 10 | 15 | 20 |
| complex 20% | | | | | | |
| Other ingredients | | | | | | |
| Chewing gum base for | 500 | 500 | 500 | 500 | 500 | 500 |
| compression | | | | | | |
| Xylitol | 258 | 252 | 243 | 224 | 206 | 188 |
| Sorbitol | 100 | 100 | 100 | 100 | 100 | 100 |
| Encapsulated | 100 | 100 | 100 | 100 | 100 | 100 |
| peppermint oil | | | | | | |
| L-Arginine | 2.9 | 5.8 | 10.8 | 21.6 | 32.4 | 43.2 |
| Sodium carbonate | 0.5 | 1 | 2.5 | 5 | 7.5 | 10 |
| Magnesium stearate | 15 | 15 | 15 | 15 | 15 | 15 |
| Talcum | 15 | 15 | 15 | 15 | 15 | 15 |
| Magnesium oxide | 5 | 5 | 5 | 5 | 5 | 5 |
| Acesulfame K | 2 | 2 | 2 | 2 | 2 | 2 |
| Aspartame | 2 | 2 | 2 | 2 | 2 | 2 |

B. Manufactured by mixing, rolling and scoring

| | 0 mg | | 1 mg | 2 mg | | |
|---|---|---|---|---|---|---|
| | Unit | 0.5 mg | Unit | Unit | 3 mg | 4 mg |
| | for- | Unit | for | for- | Unit | Unit |
| | mula | formula | mula | mula | formula | formula |
| | (mg) | (mg) | (mg) | (mg) | (mg) | (mg) |
| Active ingredient | | | | | | |
| Nicotine β- | 0 | 4.4 | 8.7 | 17.4 | 26.1 | 34.8 |
| cyclodextrin complex | | | | | | |
| 11.5% | | | | | | |
| Other ingredients | | | | | | |
| Chewing gum base | 650 | 650 | 650 | 650 | 650 | 650 |
| Xylitol | 312 | 302 | 291 | 265 | 246 | 216 |
| Peppermint oil | 30 | 30 | 30 | 30 | 30 | 30 |
| L-Arginine | 2.9 | 8.6 | 15.8 | 32.4 | 43.2 | 64.8 |
| Acesulfame K | 2 | 2 | 2 | 2 | 2 | 2 |
| Levomenthol | 2 | 2 | 2 | 2 | 2 | 2 |
| Magnesium oxide | 1 | 1 | 1 | 1 | 1 | 1 |

C. Manufactured by mixing, rolling and scoring

| | 0 mg | | 1 mg | | | |
|---|---|---|---|---|---|---|
| | Unit | 0.5 mg | Unit | 2 mg | 3 mg | 4 mg |
| | for- | Unit | for- | Unit | Unit | Unit |
| | mula | formula | mula | formula | formula | formula |
| | (mg) | (mg) | (mg) | (mg) | (mg) | (mg) |
| Active ingredient | | | | | | |
| Nicotine free base | 0 | 0.5 | 1 | 2 | 3 | 4 |
| Other ingredients | | | | | | |
| Chewing gum base | 620 | 620 | 620 | 620 | 620 | 620 |
| Xylitol | 341 | 335 | 327 | 310 | 298 | 275 |
| Peppermint oil | 30 | 30 | 30 | 30 | 30 | 30 |
| L-Arginine | 2.9 | 8.6 | 15.8 | 32.4 | 43.2 | 64.8 |
| Acesulfame K | 2 | 2 | 2 | 2 | 2 | 2 |
| Levomenthol | 2 | 2 | 2 | 2 | 2 | 2 |
| Magnesium oxide | 2 | 2 | 2 | 2 | 2 | 2 |

D. Manufactured by mixing, rolling and scoring

| | 0 mg | | 1 mg | | | |
|---|---|---|---|---|---|---|
| | Unit | 0.5 mg | Unit | 2 mg | 3 mg | 4 mg |
| | for- | Unit | for- | Unit | Unit | Unit |
| | mula | formula | mula | formula | formula | formula |
| | (mg) | (mg) | (mg) | (mg) | (mg) | (mg) |
| Active ingredient | | | | | | |
| Nicotine hydrogen | 0 | 1.7 | 3.4 | 6.8 | 10.2 | 13.6 |
| tartrate | | | | | | |
| Other ingredients | | | | | | |
| Chewing gum base | 660 | 660 | 660 | 660 | 660 | 660 |
| Xylitol | 302 | 295 | 286 | 266 | 257 | 227 |
| Fruit flavour | 30 | 30 | 30 | 30 | 30 | 30 |
| L-Arginine | 2.9 | 8.6 | 15.8 | 32.4 | 43.2 | 64.8 |
| Acesulfame K | 2 | 2 | 2 | 2 | 2 | 2 |
| Aspartame | 2 | 2 | 2 | 2 | 2 | 2 |
| Magnesium oxide | 1 | 1 | 1 | 1 | 1 | 1 |

E. Manufactured by mixing rolling and scoring

| | 0 mg | | 1 mg | | | |
|---|---|---|---|---|---|---|
| | Unit | 0.5 mg | Unit | 2 mg | 3 mg | 4 mg |
| | for- | Unit | for- | Unit | Unit | Unit |
| | mula | formula | mula | formula | formula | formula |
| | (mg) | (mg) | (mg) | (mg) | (mg) | (mg) |
| Active ingredients | | | | | | |
| Nicotine resin | 0 | 2.5 | 5 | 10 | 15 | 20 |
| complex 20% | | | | | | |
| Other ingredients | | | | | | |
| Chewing gum base | 660 | 660 | 660 | 660 | 660 | 660 |
| Xylitol | 302 | 294 | 284 | 263 | 247 | 220 |
| Peppermint oil | 30 | 30 | 30 | 30 | 30 | 30 |
| L-Arginine | 2.9 | 8.6 | 15.8 | 32.4 | 43.2 | 64.8 |
| Acesulfame K | 2 | 2 | 2 | 2 | 2 | 2 |
| Levomenthol | 2 | 2 | 2 | 2 | 2 | 2 |
| Magnesium oxide | 1 | 1 | 1 | 1 | 1 | 1 |
| Capsaicin | 25 μg | — | — | — | — | — |

### Manufacturing Procedures

I) Mixing, Rolling and Scoring

Mixing, rolling and scoring is done by a conventional procedure. Double sigma blade mixers are used for mixing the gum base with the other components of the formulation. The gum base is softened in the mixer. By heat (from the heating jacket) and mixing, the gum base becomes plastic. So, the softened base is mixed with the liquid components, e g flavours, liquid, sorbitol and glycerol, when used and the solid materials, eg nicotine in any form, buffer, bulk sweetener, colour as a powder mixture. The warm mass is discharged from the mixer in form of loaves stacked on trays on a truck and stored in a conditioned area until the next step starts. This is to cool the gum.

After this, the rolling and scoring takes place. The gum is extruded into a thick sheet, which is rolled by multiple sets of calendar rolls to the correct thickness. The scoring rolls, usually two sets, cut into the correct size.

The sheets are then transferred to a conditioned area on trays, where the sheets are cooled to make them brittle enough to be broken. The conditioned gum sheets are then passed through a breaker, which is a rotating drum that parts the sheets into separate pieces of gum along the scores.

At a sorting stage deformed gums are sorted away. The accepted gums are passed through a metal detector.

II) Compressing

Chewing gums produced by compression (usually being a dry method), ie tabletted gums, are made out of a special gum base. High velocity mixers can be used for granulation to give correctly sized particles of the mixture. This mixture is then compressed in a tablet machine.

At a sorting stage deformed gums are sorted away. The accepted gums are passed through a metal detector.

### Example 6

### Tablet Cores

This example describes without limiting the invention the manufacture of different tablet cores according to the invention.

Example 6A Directly compressible nicotine tablet (1200 mg core weight)

| | 0 mg | | 1 mg | | | |
|---|---|---|---|---|---|---|
| | Unit | 0.5 mg | Unit | 2 mg | 3 mg | 4 mg |
| | for- | Unit | for- | Unit | Unit | Unit |
| | mula | formula | mula | formula | formula | formula |
| | (mg) | (mg) | (mg) | (mg) | (mg) | (mg) |
| Active ingredients | | | | | | |
| Nicotine resin | 0 | 2.5 | 5 | 10 | 15 | 20 |
| complex 20% | | | | | | |
| Other ingredients | | | | | | |
| Mannitol | 150 | 150 | 150 | 150 | 150 | 150 |
| Xylitol | 1020 | 1015 | 1010 | 1000 | 990 | 980 |
| Mint flavor | 15 | 15 | 15 | 15 | 15 | 15 |
| Hydrogenated | 15 | 15 | 15 | 15 | 15 | 15 |
| vegetable oil | | | | | | |
| Magnesium stearate | 10 | 10 | 10 | 10 | 10 | 10 |

### Manufacturing Method:

The above ingredients are dry-blended and thereafter compressed into tablet cores. The cores are then coated using any of the methods according to Examples 1-4.

1. A coated pharmaceutical product for intraoral delivery of nicotine comprising at least one core, nicotine in any form, at least one coating layer and at least one amino acid in an amount effective to buffer said product.

2. The product according to claim 1 wherein said at least one amino acid is at least one endogenous amino acid.

3. The product according to claim 2 wherein said at least one endogenous amino acid is selected from the group consisting of Arginine, Aspargine, Glutamic acid, Glutamine, Glycine, Histidine, Isoleucine, Leucine, Lysine, Methionine, Phenylalanine, Serine, Threonine and Valine.

4. The product according to claim 1 wherein said at least one amino acid is selected from the group consisting of Cysteic acid. N-Glycylglycine and Ornithine.

5. The product according to claim 1 wherein the at least one core is selected from the group consisting of a chewing gum, a chewable tablet, a tablet, a melt tablet, a lozenge and a hard boiled candy.

6. The product according to claim 1 wherein said nicotine is in said at least one coating layer.

7. The product according to claim 1 wherein said at least one core is buffered.

8. The product according to claim 1 wherein said nicotine is in said at least one core.

9. The product according to claim 1 wherein said at least one coating layer is buffered with said amino acid and upon administration of said product to a subject the pH of the saliva of the subject is increased by 0.3-4 pH units.

10. The product according to claim 9 wherein upon administration of said product to a subject the pH of the saliva of the subject is increased by 0.5-2 pH units.

11. The product according to claim 9 wherein the pH of the saliva of the subject is increased to at least pH 7 and to at most pH 10.

12. The product according to claim 1 wherein said at least one coating layer is buffered by said at least one amino acid and further comprises a second buffering agent selected from the group consisting of a carbonate, glycinate, phosphate, glycerophosphate, acetate, gluconate, an alkali metal citrate, trometamol, and mixtures thereof.

13. The product according to claim 1 wherein said nicotine is selected from the group consisting of a nicotine salt, the free base form of nicotine, a nicotine derivative, a nicotine cation exchanger, a nicotine inclusion complex or nicotine in any non-covalent binding; nicotine bound to zeolites; nicotine bound to cellulose or starch micro-spheres; and mixtures thereof.

14. The product according to claim 13, wherein said nicotine inclusion complex is a cyclodextrin complex.

15. The product according to claim 14 wherein said nicotine inclusion complex is a cyclodextrin complex.

16. The product according to claim 13 wherein said nicotine cation exchanger is a polyacrylate cation exchanger.

17. The product according to claim 13, wherein the nicotine salt comprises a mono tartrate, hydrogen tartrate, citrate, malate or hydrochloride salt.

18. The product according to claim 1 wherein said nicotine is present in an amount from about 0.05-8 mg calculated as the free base form of nicotine per piece of coated product.

19. The product according to claim 18 wherein said nicotine is present in an amount form about of 0.1-6 mg calculated as the free base form of nicotine per piece of coated product.

20. The product according to claim 18 wherein said nicotine is present in an amount from about 0.5-5 mg calculated as the free base form of nicotine per piece of coated product.

21. The product according to claim 1 wherein said nicotine is in an amount from about 0.1-5 mg calculated as the free base form of nicotine in said at least one coating layer.

22. The product according to claim 21 wherein the nicotine is in an amount form about 0.1-3 mg calculated as the free base form of nicotine in said at least one coating layer.

23. The product according to claim 22 wherein said nicotine is in an amount form about 0.1-2 mg calculated as the free base form of nicotine in said at least one coating layer.

24. A method for delivering nicotine to a subject comprising the steps of
a) administering to a subject a coated product according to claim 1 into the oral cavity of the subject, and
b) allowing the nicotine in any form in the coated product to be released in the saliva in the oral cavity and absorbed into the systemic circulation of the subject.
